SEQUENCE LISTING (20 sequences)

SEQ ID NO: 1 - protein, 10 aa, Artificial Sequence
  Description of Artificial Sequence Synthetic HCDR1_MOR103 peptide
GFTFSSYWMN

SEQ ID NO: 2 - protein, 19 aa, Artificial Sequence
  Description of Artificial Sequence Synthetic HCDR2_MOR103 peptide
GIENKYAGGATYYAASVKG

SEQ ID NO: 3 - protein, 6 aa, Artificial Sequence
  Description of Artificial Sequence Synthetic HCDR3_MOR103 peptide
GFGTDF

SEQ ID NO: 4 - protein, 11 aa, Artificial Sequence
  Description of Artificial Sequence Synthetic LCDR1_MOR103 peptide
SGDSIGKKYAY

SEQ ID NO: 5 - protein, 5 aa, Artificial Sequence
  Description of Artificial Sequence Synthetic LCDR2_MOR103 peptide
KKRPS

SEQ ID NO: 6 - protein, 8 aa, Artificial Sequence
  Description of Artificial Sequence Synthetic LCDR3_MOR103 peptide
SAWGDKGM

SEQ ID NO: 7 - protein, 144 aa, Homo sapiens
  GM-CSF
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 8 - protein, 117 aa, Artificial Sequence [sequence omitted]

SEQ ID NO: 9 - protein, 106 aa, Artificial Sequence
  Description of Artificial Sequence Synthetic variable light chain MOR103 polypeptide
DIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 10 - protein, 297 aa, Homo sapiens
  CD20
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 11 - DNA, 351 bp, Artificial Sequence
  Description of Artificial Sequence Synthetic variable heavy chain MOR103 polynucleotide
caggtgcagctggtcgagtctggcggcggactggtgcagcctggcggcagcctgagactgagctgtgccgccagcggcttcaccttcagcagctactggatgaactgggtgaggcaggcccctggcaagggcctggagtgggtgtccggcatcgagaacaagtatgccggcggagccacctactacgccgccagcgtgaagggccggttcaccatcagccgggacaacagcaagaacaccctgtacctgcagatgaacagcctgagggccgaggacaccgccgtgtactactgtgccaggggcttcggcaccgatttctggggccagggcaccctggtgacagtcagctca

SEQ ID NO: 12 - DNA, 318 bp, Artificial Sequence
  Description of Artificial Sequence Synthetic variable light chain MOR103 polynucleotide
gacatcgagctgacccagccccccagcgtgtctgtggcccctggccagaccgcccggatcagctgctccggcgacagcatcggcaagaagtacgcctactggtatcagcagaagcccggccaggcccccgtgctggtgatctacaagaagcggcccagcggcatccccgagcggttcagcggcagcaacagcggcaacaccgccaccctgaccatcagcggcacccaggccgaggacgaggccgactactactgctccgcctggggcgacaagggcatggtgtttggcggcggaacaaagttaaccgtgctggggcag

SEQ ID NO: 13 - protein, 5 aa, Artificial Sequence
  Description of Artificial Sequence Synthetic peptide
SYNMH

SEQ ID NO: 14 - protein, 17 aa, Artificial Sequence
  Description of Artificial Sequence Synthetic peptide
AIYPGNGDTSYNQKFKG

SEQ ID NO: 15 - protein, 12 aa, Artificial Sequence
  Description of Artificial Sequence Synthetic peptide
STYYGGDWYFNV

SEQ ID NO: 16 - protein, 10 aa, Artificial Sequence
  Description of Artificial Sequence Synthetic peptide
RASSSVSYIH

SEQ ID NO: 17 - protein, 7 aa, Artificial Sequence
  Description of Artificial Sequence Synthetic peptide
ATSNLAS

SEQ ID NO: 18 - protein, 9 aa, Artificial Sequence
  Description of Artificial Sequence Synthetic peptide
QQWTSNPPT

SEQ ID NO: 19 - protein, 451 aa, Artificial Sequence
  Description of Artificial Sequence Synthetic polypeptide
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 213 aa, Artificial Sequence
  Description of Artificial Sequence Synthetic polypeptide
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2014/0234298 A1
(43) Pub. Date: Aug. 21, 2014

(54) THERAPEUTIC COMBINATIONS OF ANTI-CD20 AND ANTI-GM-CSF ANTIBODIES AND USES THEREOF

(71) Applicant: Stefan Steidl, Munchen (DE)

(72) Inventor: Stefan Steidl, Munchen (DE)

(73) Assignee: MORPHOSYS AG

(21) Appl. No.: 14/130,667

(22) PCT Filed: Jul. 6, 2012
(86) PCT No.: PCT/EP2012/063207

§ 371 Date: Apr. 23, 2014

Related U.S. Application Data
(60) Provisional application No. 61/504,744, filed on Jul. 6, 2011.

(30) Foreign Application Priority Data
Jul. 6, 2011 (EP) 11172865.5

(51) Int. Cl. C07K 16/28; C07K 16/24

(52) U.S. Cl. CPC C07K 16/2887; C07K 16/243
USPC 424/133.1; 424/139.1

(57) ABSTRACT

The present disclosure describes a pharmaceutical combination of an anti-CD20 antibody and an anti-GM-CSF antibody. Said combinations are highly efficacious in the treatment of B cell malignancies and inflammatory disorders.

### CROSS REFERENCE TO RELATED APPLICATIONS

This application claims the benefit of U.S. provisional application Ser. No. 61/504,744 filed Jul. 6, 2011, which is incorporated by reference in its entirety.

### FIELD OF THE INVENTION

The present application relates to combination therapies for the treatment of inflammatory disorders, such as rheumatoid arthritis and multiple sclerosis, and hematological disorders, such as B cell malignancies.

### BACKGROUND

### CD20

CD20 is a glycosylated phosphoprotein expressed on the surface of all mature B-cells. In humans, CD20 is encoded by the MS4A1 gene. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes a B-lymphocyte surface molecule that plays a role in the development and differentiation of B-cells into plasma cells. This family member is localized to 11q12, among a cluster of family members. Alternative splicing of this gene results in two transcript variants that encode the same protein. CD20 is expressed on all stages of B cell development except the first and last; it is present from late pro-B cells through memory cells, but not on either early pro-B cells or plasma blasts and plasma cells. It is found on B-cell lymphomas, hairy cell leukemia, B-cell chronic lymphocytic leukemia, and melanoma cancer stem cells.

CD20 is the target of several monoclonal antibodies (mAb), such as rituximab, ibritumomab tiuxetan, and tositumomab, which are all active agents in the treatment of all B cell lymphomas and leukemias. The anti-CD20 antibody ofatumumab (Genmab) was approved by FDA in October 2009 for Chronic lymphocytic leukemia. Numerous additional anti-CD20 antibody therapeutics are (or were) under development, including AME-133v (Applied Molecular Evolution), ocrelizumab (Roche, Biogen Idec), TRU-015 (Trubion), and IMMU-106 (veltuzumab; Immunomedics).

Antibody FMC7 appears to recognise a conformational variant of CD20 also known as the FMC7 antigen.

### GM-CSF

GM-CSF (Granulocyte-macrophage colony-stimulating factor) is a protein secreted by macrophages, T cells, mast cells, endothelial cells, and fibroblasts. GM-CSF is a cytokine that functions as a white blood cell growth factor. GM-CSF stimulates stem cells to produce granulocytes (neutrophils, eosinophils, and basophils) and monocytes. Monocytes exit the circulation and migrate into tissue, whereupon they mature into macrophages. Thus, it is part of the immune/inflammatory cascade, by which activation of a small number of macrophages can rapidly lead to an increase in their numbers, a process crucial for fighting infection. The active form of the protein is found extracellularly as a monomer. Human granulocyte macrophage colony-stimulating factor is glycosylated in its mature form. GM-CSF is found in high levels in joints with rheumatoid arthritis and blocking GM-CSF may reduce the inflammation or damage.

Some therapeutics (eg MOR103) are being developed to block GM-CSF, e.g. MOR103 (MorphoSys), an anti-GM-CSF mAb. Other anti-GM-CSF antibody therapeutics under development include KB002 and KB003 (KaloBios) and MT203 (Micromet and Nycomed). Other companies develop or have developed anti-GM-CSF antibodies as well, e.g. Morphotek, Evec, Boehringer Ingelheim and Amgen.

### Combination Therapy

Although anti-CD20 mAb's and anti-GM-CSF mAb's are used individually, or in combination therapy with other agents, they have never been used together in the treatment of disease. Anti-GM-CSF mAb's are in development for the treatment of inflammatory disorders. Anti-CD20 mAb's are primarily used for the treatment of B cell malignancies, but also for rheumatoid arthritis. Additionally, anti-CD20 mAb's show promising results in clinical trials for multiple sclerosis. Nevertheless, novel and superior treatments are still urgently needed for patients afflicted with the aforementioned diseases and disorders. Certain disclosures cursorly mention a potential combination treatment of anti-CD20 mAb's with GM-CSF or peptides derived from GM-CSF, e.g. WO2010115554, WO2000027428; WO2000047228 and WO2003068821, but none exemplify a combination with anti-GM-CSF mAb's.

Sakagami et al. (Am J Respir Crit Care Med (2010) 182, 49-61) report that anti-GM-CSF autoantiobodies are able to reproduce the molecular, cellular, and histological features of pulmonary alveolar proteinosis (PAP) in healthy animals. Sakagami et al. utilize polyclonal GM-CSF autoantibodies isolated from biopsy-proven patients with PAP. Sakagami et al. do not report or suggest treatment with anti-GM-CSF antibodies, but to the contrary show that such anti-GM-CSF antibodies are capaple of causing or inducing certain diseases or symptoms, i.e. PAP. Sakagami et al. therefore does not disclose the treatment of any disease with anti-GM-CSF antibodies, in particular not with any monoclonal anti-GM-CSF antibodies. In their manuscript Sakagami et al. also report the coincidental finding that anti-CD20 mediated B cell depletion is strongly enhanced and B cell reconstitution is strongly suppressed in the presence of such anti-GM-CSF autoantibodies. There is currently no mechanistic theory however, that could explain such a finding.

Additionally, there are reports that contradict the usefulness of an anti-CD20-anti-GM-CSF combination therapy. See Kavuru et al. (Eur Respir J. 2011 38:1361-7) and Vallerskog et al. (Clin Immunol (2007) 122, 62-74). Both show similar levels of B-cell depletion upon treatment with an anti-CD20 mAb, despite the fact that in Kavuru the anti-CD20 mAb was used in the presence of anti-GM-CSF antibodies. This finding suggests that the presence of anti-GM-CSF antibodies does not increase the effectiveness of an anti-CD20 mAb in depleting B-cells. Kavuru et al. shows that B cells are depleted upon treatment with rituximab, an anti-CD20 mAb, in patients with idiopathic pulmonary alveolar proteinosis (PAP). PAP is characterized by the presence of anti-GM-CSF antibodies. Recovery of the B-cell population was observed around 6 months post-treatment. Vallerskog et al. investigated B cell depletion upon treatment with rituximab in patients with systemic lupus erythematosus (SLE). They found B-cell recovery also around 6 months post-treatment. In contrast to PAP, SLE patients are, however, not characterized by anti-GM-CSF autoantibodies. The similar results obtained in the studies from Kavuru et al. and Vallerskog et al. therefore rather suggest that the combination of an anti-CD20 and anti-GM-CSF antibody does not increase B cell depletion compared to an anti-CD20 mAb alone.

### SUMMARY OF THE INVENTION

In certain aspects the present invention relates to a synergistic combination of an antibody specific for CD20 and an antibody specific for GM-CSF for use in medicine. In certain preferred aspect said antibodies specific for CD20 and specific for GM-CSF are monoclonal antibodies.

Said synergistic combination may be used in the treatment of B cell malignancies, including non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma).

Said synergistic combination may also be used in the treatment of an inflammatory disorder, including ulcerative colitis, Crohn's disease, inflammatory bowel disease, rheumatoid arthritis, myositis, multiple sclerosis, neuromyelitis optica, atherosclerosis, psoriasis, systemic lupus erythematosus, nephritis, glomerulonephritis, autoimmune hepatobiliary disease, graft-versus-host disease, atopic dermatitis, asthma, neurodegenerative disease (e.g., Alzheimer's disease), demyelinating polyradiculopathy, neuropathic pain, atherosclerosis, age-related macular degeneration, diabetic nephropathy, sarcoidosis-origined uveitis, or diabetes mellitus.

In certain aspects the components of the synergistic combination of the present invention are administered separately. The antibody specific for CD20 may be administered prior to the antibody specific for GM-CSF. Alternatively, the antibody specific for GM-CSF may be administered prior to the antibody specific for CD20. In certain aspects the components of the synergistic combination of the present invention are administered simultaneously or at about the same time.

Any antibody specific for CD20 may be used to practice the present invention, including Rituximab, Ibritumomab, Tositumomab, Bexxar, Ofatumumab, Ocrelizumab, BLX-301, Veltuzumab, DXL625 or any other antibody specific for CD20 mentioned in the present invention or known in the art. Likewise, any antibody specific for GM-CSF may be used to practice the present invention, including MOR103 or any one of the anti-GM-CSF antibodies disclosed in WO2006111353, US20090297532, WO2007049472, US20080317757, WO2009064399, US20100122819, WO2003068920, US20040053365, WO2007092939, US20080292641, WO2008141391, US20100297135, WO2009038760, U.S. Ser. No. 12/675,013, WO2009062238, US20100297135, WO2009134805, US20090274706, WO2010124163, US20100291075 or any other antibody specific for GM-CSF mentioned in the present invention or known in the art. In certain aspect the antibody specific for GM-CSFcomprises an HCDR1 region of sequence GFTFSSYWMN, an HCDR2 region of sequence GIENKYAGGATYYAASVKG, an HCDR3 region of sequence GFGTDF, an LCDR1 region of sequence SGDSIGKKYAY, an LCDR2 region of sequence KKRPS, and an LCDR3 region of sequence SAWGDKGM.

### BRIEF DESCRIPTION OF DRAWINGS

FIG. 1 shows the amino acid sequence and the DNA sequence of MOR04357.

### DESCRIPTION OF THE INVENTION

“Synergy”, “synergism” or “synergistic” mean more than the expected additive effect of a combination. The “synergy”, “synergism” or “synergistic” effect of a combination is determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol Rev 58:621-681 (2006), which is incorporated by reference in its entirety. See also Clarke et al., Issues in experimental design and endpoint analysis in the study of experimental cytotoxic agents in vivo in breast cancer and other models, Breast Cancer Research and Treatment 46:255-278 (1997), which is incorporated by reference in its entirety.

The term “antibody” means monoclonal antibodies, including any isotype, such as, IgG, IgM, IgA, IgD and IgE. An IgG antibody is comprised of two identical heavy chains and two identical light chains that are joined by disulfide bonds. Each heavy and light chain contains a constant region and a variable region. Each variable region contains three segments called “complementarity-determining regions” (“CDRs”) or “hypervariable regions”, which are primarily responsible for binding an epitope of an antigen. They are referred to as CDR1, CDR2, and CDR3, numbered sequentially from the N-terminus. The more highly conserved portions of the variable regions outside of the CDRs are called the “framework regions”. An “antibody fragment” means an Fv, scFv, dsFv, Fab, Fab′F(ab′)2 fragment, or other fragment, which contains at least one variable heavy or variable light chain, each containing CDRs and framework regions.

The term “monoclonal” is to be understood as having the meaning typically ascribed to it in the art, namely an antibody or an antibody fragment arising from a single clone of an antibody-producing cell, such as a B cell, and recognizing a single epitope on the antigen bound.

“VH” refers to the variable region of an immunoglobulin heavy chain of an antibody, or antibody fragment. “VL” refers to the variable region of the immunoglobulin light chain of an antibody, or antibody fragment.

The “CDRs” herein are defined by either Chothia et al or Kabat et al. See Chothia C, Lesk A M. (1987) Canonical structures for the hypervariable regions of immunoglobulins. J Mol Biol., 196(4):901-17, which is incorporated by reference in its entirety. See Kabat E. A, Wu T. T., Perry H. M., Gottesman K. S. and Foeller C. (1991). Sequences of Proteins of Immunological Interest. 5th edit., NIH Publication no. 91-3242, US Dept. of Health and Human Services, Washington, D.C., which is incorporated by reference in its entirety.

The terms “GM-CSF” and “GMCSF” refer to the protein known as GM-CSF or Granulocyte-macrophage colony-stimulating factor, having the following synonyms: Colony-stimulating factor 2, CSF2, GMCSF, GM-CSF, Granulocyte-macrophage colony-stimulating factor, MGC131935, MGC138897, Molgramostin, Sargramostim. Human GM-CSF has the amino acid sequence of (UniProt P04141):

MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE

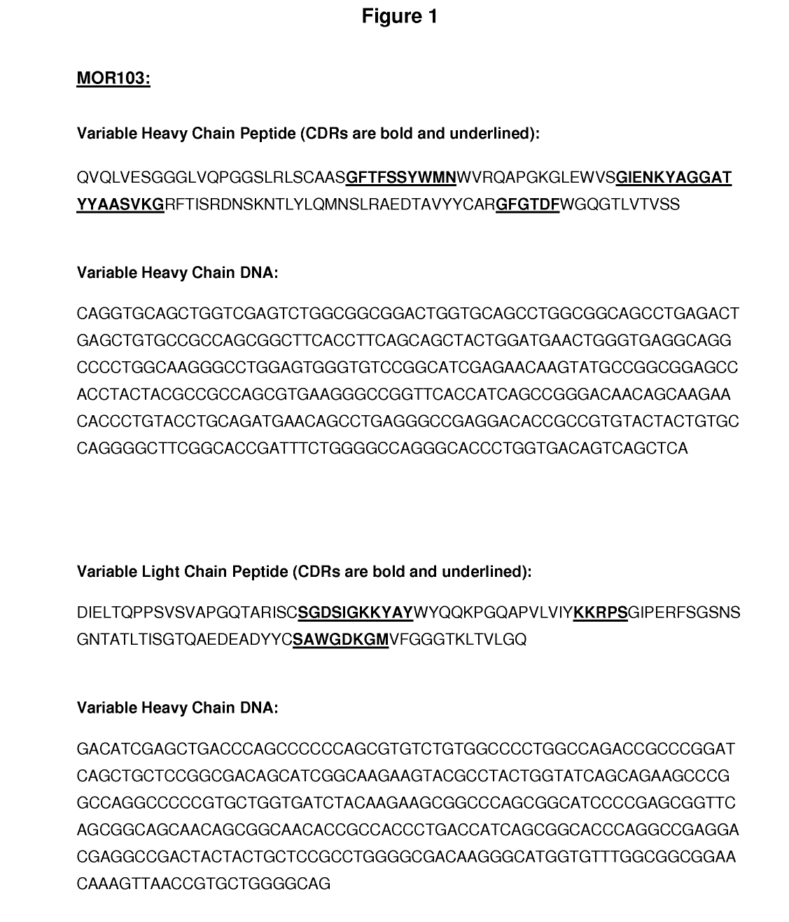

“MOR103” is an anti-GM-CSF antibody whose amino acid sequence and DNA sequence is provided in FIG. 1. “MOR103” and “MOR04357” and “MOR4357” are used as synonyms to describe the antibody shown in FIG. 1. MOR04357 comprises an HCDR1 region of sequence GFTFSSYWMN, an HCDR2 region of sequence GIENKYAGGATYYAASVKG, an HCDR3 region of sequence GFGTDF, an LCDR1 region of sequence SGDSIGKKYAY, an LCDR2 region of sequence KKRPS, and an LCDR3 region of sequence SAWGDKGM. MOR04357 comprises a variable heavy chain of the sequence

QVQLVESGGGLVQPGGSLRLSCAASGFTFSSYWMNWVRQAPGKGLEWVSGIENKYA GGATYYAASVKGRFTISRDNSKNTLYLQMNSLRAEDTAVYYCARGFGTDFWGQGTLV TVSS and a variable light chain of the sequence DIELTQPPSVSVAPGQTARISCSGDSIGKKYAYWYQQKPGQAPVLVIYKKRPSGIPERF SGSNSGNTATLTISGTQAEDEADYYCSAWGDKGMVFGGGTKLTVLGQ.

In certain embodiments the antibody specific for GM-CSF is an antibody which cross-competes with an antibody specific for GM-CSF comprising an HCDR1 region of sequence GFTFSSYWMN, an HCDR2 region of sequence GIENKYAGGATYYAASVKG, an HCDR3 region of sequence GFGTDF, an LCDR1 region of sequence SGDSIGKKYAY, an LCDR2 region of sequence KKRPS, and an LCDR3 region of sequence SAWGDKGM.

In certain embodiments the antibody specific for GM-CSF is an antibody which binds to the same epitope like an antibody specific for GM-CSF comprising an HCDR1 region of sequence GFTFSSYWMN, an HCDR2 region of sequence GIENKYAGGATYYAASVKG, an HCDR3 region of sequence GFGTDF, an LCDR1 region of sequence SGDSIGKKYAY, an LCDR2 region of sequence KKRPS, and an LCDR3 region of sequence SAWGDKGM.

In certain embodiments the present invention provides a synergistic combination an antibody specific for CD 20 and an antibody specific for GM-CSF, wherein the antibody specific for GM-CSF comprises an HCDR1 region of sequence GFTFSSYWMN, an HCDR2 region of sequence GIENKYAGGATYYAASVKG, an HCDR3 region of sequence GFGTDF, an LCDR1 region of sequence SGDSIGKKYAY, an LCDR2 region of sequence KKRPS, and an LCDR3 region of sequence SAWGDKGM.

In certain embodiments the present invention provides a synergistic combination an antibody specific for CD 20 and an antibody specific for GM-CSF, wherein the antibody specific for GM-CSF comprises a variable heavy chain of the sequence QVQLVESGGGLVQPGGSLRLSCAASGFTFSSYWMNWVRQAPGKGLEWVSGIENKYA GGATYYAASVKGRFTISRDNSKNTLYLQMNSLRAEDTAVYYCARG FGTDFWGQGTLV TVSS and a variable light chain of the sequence DIELTQPPSVSVAPGQTARISCSGDSIGKKYAYWYQQKPGQAPVLVIYKKRPSGIPERF SGSNSGNTATLTISGTQAEDEADYYCSAWG DKGMVFGGGTKLTVLGQ.

Antibodies specific for GM-CSF include namilumab (MT-203), a fully-human IgG1 against GM-CSF developed by Micromet (now Amgen), MORAb-022 is a fully-human mAb targeting GM-CSF developed by Morphotek (Eisai) and the GM-CSF antibodies derived from human IgG memory B-cells (Theraclone Sciences, formerly Spaltudaq). Other antibodies specific for GM-CSF are described in WO2006111353 (U.S. Ser. No. 11/918,368, expressly incorporated herein by reference) (Micromet), WO2007049472 (U.S. Ser. No. 12/149,009, expressly incorporated herein by reference) (Evec), WO2009064399, (U.S. Ser. No. 12/681,396, expressly incorporated herein by reference) (Evec, Boehringer Ingelheim), WO2003068920 (U.S. Ser. No. 10/365,123, expressly incorporated herein by reference) (Ludwig Institute for Cancer Research), WO2007092939 (U.S. Ser. No. 11/672,902, expressly incorporated herein by reference) (Morphotek), WO2008141391 (U.S. Ser. No. 12/601,514, expressly incorporated herein by reference) (CRC for Asthma and Airways), WO2009038760 (U.S. Ser. No. 12/675,013, expressly incorporated herein by reference) (Amgen), WO2009062238 (U.S. Ser. No. 12/742,467, expressly incorporated herein by reference) (CRC for Asthma and Airways), WO2009134805 (U.S. Ser. No. 12/431,661, expressly incorporated herein by reference) (Kalobios) and WO2010124163 (U.S. Ser. No. 12/766,444, expressly incorporated herein by reference) (Theraclone). All antibodies disclosed in aforementioned patents and patent applications may be used within the present invention.

Some of the antibodies disclosed in aforementioned patents also cursorily mention combination therapies in a laundry list type fashion. None however discloses a combination therapy with anti-GM-CSF antibodies. A specific combination therapy of an antibody specific for GM-CSF with IL17 antagonists is disclosed in WO 2009/133103 (Micromet).

The term “CD20” refers to the protein known as CD20 or MS4A1, having the following synonyms: B1, B-lymphocyte antigen CD20, B-lymphocyte surface antigen B1, Bp35, CD20, CVID5, LEU-16, Leukocyte surface antigen Leu-16, Membrane-spanning 4-domains subfamily A member 1, MGC3969, MS4A2, S7. Human CD20 has the amino acid sequence of (UniProt P011836):

MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP

Examples of antibodies specific for CD20 antigen include: “C2B8” which is now called “Rituximab” (“RITUXAN®”) (U.S. Pat. No. 5,736,137, expressly incorporated herein by reference), a chimaeric pan-B antibody targeting CD20; the yttrium-[90]-labeled 2B8 murine antibody designated “Y2B8” or “Ibritumomab Tiuxetan” ZEVALIN® (U.S. Pat. No. 5,736,137, expressly incorporated herein by reference), a murine IgG1 kappa mAb covalently linked to MX-DTPA for chelating to yttrium-[90]; murine IgG2a “BI,” also called “Tositumomab,” optionally labeled with radioactive 131I to generate the “1311-B1” antibody (iodine 131 tositumomab, BEXXAR™) (U.S. Pat. No. 5,595,721, expressly incorporated herein by reference); murine monoclonal antibody “1F5” (Press et al. Blood 69 (2):584-591 (1987) and variants thereof including “framework patched” or humanized 1F5 (WO03/002607, Leung, S.; ATCC deposit HB-96450); murine 2H7 and chimeric 2H7 antibody (U.S. Pat. No. 5,677,180, expressly incorporated herein by reference); humanized 2H7, also known as ocrelizumab (PRO-70769); Ofatumumab (Arzerra), a fully human IgG1 against a novel epitope on CD20 huMax-CD20 (Genmab, Denmark; WO2004/035607 (U.S. Ser. No. 10/687,799, expressly incorporated herein by reference)); AME-133 (ocaratuzumab; Applied Molecular Evolution), a a fully-humanized and optimized IgG1 mAb against CD20; A20 antibody or variants thereof such as chimeric or humanized A20 antibody (cA20, hA20, respectively) (U.S. Ser. No. 10/366,709, expressly incorporated herein by reference, Immunomedics); and monoclonal antibodies L27, G28-2, 93-1B3, B-CI or NU-B2 available from the International Leukocyte Typing Workshop (Valentine et al, In: Leukocyte Typing III (McMichael, Ed., p. 440, Oxford University Press (1987)). Further, suitable antibodies include e.g. antibody GA101 (obinutuzumab), a third generation humanized anti-CD20-antibody of Biogen Idec/Genentech/Roche. Moreover, BLX-301 of Biolex Therapeutics, a humanized anti CD20 with optimized glycosylation or Veltuzumab (hA20), a 2nd-generation humanized anti-CD20 antibody of Immunomedics or DXL625, derivatives of veltuzumab, such as the bispecific hexavalent antibodies of IBC Pharmaceuticals (Immunomedics) which are comprised of a divalent anti-CD20 IgG of veltuzumab and a pair of stabilized dimers of Fab derived from milatuzumab, an anti-CD20 mAb enhanced with InNexus' Dynamic Cross Linking technology, of Inexus Biotechnology both are humanized anti-CD20 antibodies are suitable. Further suitable antibodies are BM-ca (a humanized anti-CD20 antibody (Int J. Oncol. 2011 February; 38(2):335-44)), C2H7 (a chimeric anti-CD20 antibody (Mol Immunol. 2008 May; 45(10):2861-8)), PRO131921 (a third generation anti-CD20 antibody developed by Genentech), Reditux (a biosimilar version of rituximab developed by Dr Reddy's), PBO-326 (a biosimilar version of rituximab developed by Probiomed), a biosimilar version of rituximab developed by Zenotech, TL-011 (a biosimilar version of rituximab developed by Teva), CMAB304 (a biosimilar version of rituximab developed by Shanghai CP Guojian), GP-2013 (a biosimilar version of rituximab developed by Sandoz (Novartis)), SAIT-101 (a biosimilar version of rituximab developed by Samsung BioLogics), a biosimilar version of rituximab developed by Intas Biopharmaceuticals, CT-P10), a biosimilar version of rituximab developed by Celltrion), a biosimilar version of rituximab developed by Biocad, Ublituximab (LFB-R603, a transgenically produced mAb targeting CD20 developed by GTC Biotherapeutics (LFB Biotechnologies)), PF-05280586 (presumed to be a biosimilar version of rituximab developed by Pfizer), Lymphomun (Bi-20, a trifunctional anti-CD20 and anti-CD3 antibody, developed by Trion Pharma), a biosimilar version of rituximab developed by Natco Pharma, a biosimilar version of rituximab developed by iBio, a biosimilar version of rituximab developed by Gedeon Richter/Stada, a biosimilar version of rituximab developed by Curaxys, a biosimilar version of rituximab developed by Coherus Biosciences/Daiichi Sankyo, a biosimilar version of rituximab developed by BioXpress, BT-D004 (a biosimilar version of rituximab developed by Protheon), AP-052 (a biosimilar version of rituximab developed by Aprogen), a biosimilar version of ofatumumab developed by BioXpress, MG-1106 (a biosimilar version of rituximab developed by Green Cross), IBI-301 (a humanized monoclonal antibody against CD20 developed by Innovent Biologics), BVX-20 (a humanized mAb against the CD20 developed by Vaccinex), 20-C2-2b (a bispecific mAb-IFNalpha that targets CD20 and human leukocyte antigen-DR (HLA-DR) developed by Immunomedics), MEDI-552 (developed by MedImmune/AstraZeneca), the anti-CD20/streptavidin conjugates developed by NeoRx (now Poniard Pharmaceuticals), the 2nd generation anti-CD20 human antibodies developed by Favrille (now MMRGlobal), TRU-015, an anti-CD20 antibody fragment developed by Trubion/Emergent BioSolutions, as well as other precloinical approaches by various companies and entities. All aforementioned publications, references, patents and patent applications are incorporated by reference in their entireties. All antibodies disclosed in therein may be used within the present invention.

In certain preferred embodiments of the present invention said antibody specific for CD20 is rituxan. Rituxan comprises an HCDR1 region of sequence SYNMH, an HCDR2 region of sequence AIYPGNGDTSYNQKFKG, an HCDR3 region of sequence STYYGGDWYFNV, an LCDR1 region of sequence RASSSVSYIH, an LCDR2 region of sequence ATSNLAS, and an LCDR3 region of sequence QQWTSNPPT. Rituxan comprises a variable heavy chain of the sequence:

QVQLQQPGAELVKPGASVKMSCKASGYTFTSYNMHWVKQTPGRGLEWI
GAIYPGNGDTSYNQKFKGKATLTADKSSSTAYMQLSSLTSEDSAVYYCAR
STYYGGDWYFNVWGAGTTVTVSAASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSL
SPGK,

and a variable light chain of the sequence:

QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLS
SPVTKSFNRGEC.

In certain embodiments the antibody specific for CD20 is an antibody which cross-competes with an antibody specific for CD20 comprising an HCDR1 region of sequence SYNMH, an HCDR2 region of sequence AIYPGNGDTSYNQKFKG, an HCDR3 region of sequence STYYGGDWYFNV, an LCDR1 region of sequence RASSSVSYIH, an LCDR2 region of sequence ATSNLAS, and an LCDR3 region of sequence QQWTSNPPT.

In certain embodiments the antibody specific for CD20 is an antibody which binds to the same epitope like an antibody specific for CD20 comprising an HCDR1 region of sequence SYNMH, an HCDR2 region of sequence AIYPGNGDTSYNQKFKG, an HCDR3 region of sequence STYYGGDWYFNV, an LCDR1 region of sequence RASSSVSYIH, an LCDR2 region of sequence ATSNLAS, and an LCDR3 region of sequence QQWTSNPPT.

A “combination” means more than one item, e.g. a compound such as an antibody specific for CD20 and an antibody specific for GM-CSF.

The present disclosure also relates to combinations, pharmaceuticals, and pharmaceutical compositions containing the described combinations. The two components of the synergistic combination of the present invention, i.e. the antibody specific for CD20 and the antibody specific for GM-CSF the, may be administered together, or separately. When administered together, the two components may be formulated together in one pharmaceutical composition, which may include a pharmaceutical acceptable carrier or excipient. Alternatively the two components might also be formulated in different pharmaceutical compositions. Therefore, in certain embodiments of the present invention the synergistic combination comprising an antibody specific for CD20 and an antibody specific for GM-CSF are administered separately. In this case the two components can be administered simultaneously or subsequently.

In certain preferred embodiments of the present inventions said antibody specific for CD20 is a monoclonal antibody. In other preferred embodiments of the present inventions said antibody specific for GM-CSF is a monoclonal antibody. In a most preferred embodiment of the present inventions said antibody specific for CD20 and said antibody specific for GM-CSF are monoclonal antibodies.

In certain embodiments of the present inventions said synergistic combination of the present invention comprises an antibody specific for CD20, wherein said antibody specific for CD20 is selected from Rituximab, Ibritumomab, Tositumomab, Bexxar, Ofatumumab, Ocrelizumab, BLX-301, Veltuzumab and DXL625. In preferred embodiments said antibody specific for CD20 is rituxan.

In certain embodiments of the present inventions said synergistic of the present invention comprises an antibody specific for GM-CSF, wherein said antibody specific for GM-CSF is selected from MOR103 or any one of the anti-GM-CSF antibodies disclosed in WO2006111353, WO2007049472, WO2009064399, WO2003068920, WO2007092939, WO2008141391, WO2009038760, WO2009062238, WO2009134805 or WO2010124163.

In certain embodiments of the present invention the antibody specific for CD20 is administered prior to the antibody specific for GM-CSF. In other embodiments of the present invention the antibody specific for GM-CSF is administered prior to the antibody specific for CD20.

In yet other embodiments of the present invention the antibody specific for GM-CSF and the antibody specific for CD20 are administered simultaneously. In this context the term “simultaneously” refers to a situation in which the two compositions are administered at about the same time, i.e. at the same time or immediately after each other (e.g. one injection comprising the first antibody is given immediately before the second injection comprising the second antibody).

A pharmaceutical composition includes an active agent, eg. an antibody for therapeutic use in humans. A pharmaceutical composition may include acceptable carriers or excipients.

“Administered” or “administration” includes but is not limited to delivery by an injectable form, such as, for example, an intravenous, intramuscular, intradermal or subcutaneous route or mucosal route, for example, as a nasal spray or aerosol for inhalation or as an ingestable solution, capsule or tablet.

A “therapeutically effective amount” of a compound or combination refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist.

“B-cell malignancy” includes any type of leukemia or lymphoma of B cells. B-cell malignancies include, but are not limited to, non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL) and related lymphomas (e.g. Waldenstrom's macroglobulinaemia (also called lymphoplasmacytic lymphoma or immunocytoma) or central nervous system lymphomas), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). Additional B cell malignancies include small lymphocytic lymphoma, B cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B cell lymphoma of mucosa-associated (MALT) lymphoid tissue, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma/leukemia, grey zone lymphoma, B cell proliferations of uncertain malignant potential, lymphomatoid granulomatosis, and post-transplant lymphoproliferative disorder.

In certain embodiments of the present invention the synergistic combination of the present invention is used in the treatment of B cell malignancies. In other embodiments said B cell malignancy is selected from non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma).

An “inflammatory disorder” as used herein refers to any disease, disorder, or condition in which the immune system is abnormally activated. The inflammatory disorder may be, e.g., ulcerative colitis, Crohn's disease, inflammatory bowel disease, rheumatoid arthritis, myositis, multiple sclerosis, neuromyelitis optica, atherosclerosis, psoriasis, systemic lupus erythematosus (e.g., lupus of the central nervous system or lupus nephritis), nephritis, glomerulonephritis, autoimmune hepatobiliary disease (e.g., autoimmune hepatitis, primary biliary cirrhosis, or primary sclerosing cholangitis), graft-versus-host disease, atopic dermatitis, asthma, neurodegenerative disease (e.g., Alzheimer's disease), demyelinating polyradiculopathy (e.g., Guillain-Barre syndrome or chronic inflammatory demyelinating polyradiculopathy), neuropathic pain, visceral pain of cancer, atherosclerosis, age-related macular degeneration, diabetic nephropathy, sarcoidosis-origined uveitis, or diabetes mellitus.

In certain embodiments of the present invention the synergistic combination of the present invention is used in the treatment of an inflammatory disorder. In other embodiments said inflammatory disorder is selected from ulcerative colitis, Crohn's disease, inflammatory bowel disease, rheumatoid arthritis, myositis, multiple sclerosis, neuromyelitis optica, atherosclerosis, psoriasis, systemic lupus erythematosus, nephritis, glomerulonephritis, autoimmune hepatobiliary disease, graft-versus-host disease, atopic dermatitis, asthma, neurodegenerative disease (e.g., Alzheimer's disease), demyelinating polyradiculopathy, neuropathic pain, atherosclerosis, age-related macular degeneration, diabetic nephropathy, sarcoidosis-origined uveitis, or diabetes mellitus.

In certain embodiments the present invention provides a method for the treatment of a patient with a synergistic combination of an antibody specific for CD20 and an antibody specific for GM-CSF. In certain embodiments said treatment of a patient is the treatment of a B cell malignancy, for example a B cell malignancy selected from non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma). In other embodiments said treatment of a patient is the treatment of an inflammatory disorder, for example an inflammatory disorder selected from ulcerative colitis, Crohn's disease, inflammatory bowel disease, rheumatoid arthritis, myositis, multiple sclerosis, neuromyelitis optica, atherosclerosis, psoriasis, systemic lupus erythematosus, nephritis, glomerulonephritis, autoimmune hepatobiliary disease, graft-versus-host disease, atopic dermatitis, asthma, neurodegenerative disease (e.g., Alzheimer's disease), demyelinating polyradiculopathy, neuropathic pain, atherosclerosis, age-related macular degeneration, diabetic nephropathy, sarcoidosis-origined uveitis, or diabetes mellitus.

In vitro and in vivo models are considered predictive of how a certain compound or combination of compounds will behave in humans. Here, the combination of an antibody specific for CD20 and an antibody specific for GM-CSF is tested in relevant models. When compounds are combined either in vitro or in vivo, one expects that the combination has only additive effects. Unexpectedly, the inventors found that the combination of an antibody specific for CD20 and an antibody specific for GM-CSF shows syngistic activity. The combination of the two antibodies is significantly stronger than the individual activities of each of the antibodies alone, and also significantly stronger than the expected, calculated activity of the combination. The synergistic effect of the combination will be useful in the treatment of all disease and disorders in which the synergistic combination will be used clinically. This includes the indications recited above, i.e. B cell malignancies and inflammatory disorders.

### EXAMPLES

### Example 1

### Generation of a GM-CSF Deficient (GM-CSF^{-/-} Mouse

The generation of GM-CSF−/− mice is described in Stanley et al (1994). Proc. Natl. Acad. Sci. USA 91:5592. Briefly, chimeric mice were generated by microinjection of 129/OLA-derived ES cells (H-2b) with a disrupted GM-CSF gene into C57BL/6 (H-2b) host blastocysts. Germline transmitters of the mutated GM-CSF allele were crossed with C57BL/6 mice for 11 generations, giving GM-CSF+/− mice that were interbred to yield the GM-CSF−/−, GM-CSF+/−, and GM-CSF+/+ mice used for the experiments. GM-CSF genotype status was determined by PCR analysis of tail DNA. Animals were fed standard rodent chow and water ad libitum and were housed with same sex littermates in sawdust-lined cages. Mice of both sexes were consigned to experiments at 8 to 15 wk of age.

### Example 2

### In Vivo Experiment: B-Cell Depletion in a GM-CSF^{-/-} Mouse

In this experiment we demonstrate the effect of anti-CD20 antibodies on B-cell depletion in a GM-CSF−/− knock-out mouse. GM-CSF−/− knock-out mice and wild-type strain control mice are both treated with 3 weekly doses of 250 μg (i.p) of an anti-murine CD20 IgG2a antibody (clone 18B12; see US 20070136826).

B-cell populations obtained from peripheral blood and spleen of both mouse strains are recovered at various time points after treatment with anti-CD20 antibodies and monitored by flow cytometry for CD22 and CD19 positivity. For both mouse strains B-cells are depleted, in peripheral blood and spleen however, B-cell depletion is sustained for a significantly longer time period in the GM-CSF−/− mice as compared to the wild type C57BL/6 control mice in both compartments.

This indicates that the combined depletion of both, GM-CSF and CD20, leads to a statistically significantly prolonged depletion of B-cells.

### Example 3

### In Vivo Experiment: B-Cell Depletion in a Model of B-Cell Lymphoma

5×10E6 CD20-positive murine B-lymphoma cells (BL3750; isolated as described in Minard-Colin et al. (Blood (2008) 112, 1205-13)) are s.c. inoculated in the abdomen of immunocompetent syngeneic C57BL/6 mice. Mice are then divided into four different treatment groups (10-15 mice per group) for treatment on day 3 post tumor inoculation:
Group 1: Control group; isotype control antibody (mouseIgG2a)
Group 2: anti-mouse CD20 antibody (mouseIgG2a; clone 18B12)
Group 3: anti-mouse GM-CSF antibody (rat IgG2a, clone 22E9)
Group 4: anti-mouse CD20 clone 18B12 and anti-mouse GM-CSF antibody clone 22E9

Mice are then treated with the indicated antibody (250 μg/dose weekly). An anti mouse-CD20 antibody is used, e.g. any CD20 antibody cross reactive with mouse CD20 leading to B-cell depletion via antibody effector functions. Here, as an exemplary anti-mouse GM-CSF antibody we use 22E9, a rat anti-mouse GM-CSF-specific antibody of IgG2a isotype. 22E9 is purchased from AbD Serotec (Martinsried, Germany; Cat. No. 1023501). Alternative suppliers exist, e.g. eBioscience (SanDiego, Calif., USA, Cat. No. 14-7331).

The mice treated with both antibodies, i.e. mice of Group 4, show a statistically significant delay in tumor growth and an increase in survival time as compared to the other treatment groups. This demonstrates that an anti-CD20-anti-GM-CSF combination therapy is highly and significantly more efficacious than any of the respective monotherapies.

### Example 4

### In Vivo Experiment: B-Cell Depletion in a Cynomolgus Monkeys

All cynomolgus monkeys are treated with two sequential doses of an anti-CD20 human IgG1 antibody (Rituximab) i.v. at 10 μg/kg on day 1 and 1000 μg/kg on day 3. Animals of treatment group 1 additionally receive co-administration of a neutralizing human IgG1 anti-GM-CSF antibody (MOR103; 5000 μg/kg i.v.) on day 1, while the control group 2 receives saline with the same injection volume.

B-cell populations of both groups of cynomolgus monkeys are recovered at various time points after treatment and monitored by flow cytometry. To this end venous blood samples were collected via femoral veins. B-cell counts were determined by FACS. Lymphocytes were identified and gated by light scatter, and the changes in frequency of CD19-positive B cells in the lymphocyte gate were measured.

For both treatment groups B-cells are depleted, however, B-cell depletion is sustained for a significantly longer time period in the cynomolgus monkey group treated with both antibodies, i.e. the anti-CD20 antibody and the anti-GM-CSF antibody, as compared to the group treated with the anti-CD20 antibody only.

This indicates that the combined depletion of both, GM-CSF and CD20, leads to a statistically significant prolonged depletion of B-cells.

### Example 5

### ELISA-Based Cross-Competition Assay

Cross-competition of an anti-CD20 antibody or another CD20 binding agent may be detected by using an ELISA assay according to the following standard procedure. Likewise, cross-competition of an anti-GM-CSF antibody or another GM-CSF binding agent may be detected.

The general principle of the ELISA-assay involves coating an anti-CD20 (or anti-GM-CSF) antibody onto the wells of an ELISA plate. An excess amount of a second, potentially cross-competitive, anti-CD20 (or anti-GM-CSF) antibody is then added in solution (i.e. not bound to the ELISA plate). Subsequently a limited amount of CD20-Fc (or GM-CSF-Fc) is then added to the wells.

The antibody which is coated onto the wells and the antibody in solution will compete for binding of the limited number of CD20 (or GM-CSF) molecules. The plate is then washed to remove CD20 (GM-CSF) molecules that has not bound to the coated antibody and to also remove the second, solution phase, antibody as well as any complexes formed between the second, solution phase antibody and CD20 (GM-CSF). The amount of bound CD20 (GM-CSF) is then measured using an appropriate CD20 (GM-CSF) detection reagent. Therefore, CD20 (GM-CSF) may be fuesd with a tag, like e.g. Fc, Flag, etc. which can be detected via an appropriate tag-specific antibody.

An antibody in solution that is cross-competitive to the coated antibody will be able to cause a decrease in the number of CD20 (GM-CSF) molecules that the coated antibody can bind relative to the number of CD20 (GM-CSF) molecules that the coated antibody can bind in the absence of the second, solution phase antibody.

This assay is described in more detail further below for two antibodies termed Ab-X and Ab-Y. In the instance where Ab-X is chosen to be the immobilized antibody, it is coated onto the wells of the ELISA plate, after which the plates are blocked with a suitable blocking solution to minimize non-specific binding of reagents that are subsequently added. An excess amount of Ab-Y is then added to the ELISA plate such that the moles of Ab-Y CD20 (GM-CSF) binding sites per well are at least 10 fold higher than the moles of Ab-X CD20 (GM-CSF) binding sites that are used, per well, during the coating of the ELISA plate. CD20 (GM-CSF) is then added such that the moles of CD20 (GM-CSF) added per well were at least 25-fold lower than the moles of Ab-X CD20 (GM-CSF) binding sites that are used for coating each well. Following a suitable incubation period, the ELISA plate is washed and a CD20 (GM-CSF) detection reagent is added to measure the amount of CD20 (GM-CSF) molecules specifically bound by the coated anti-CD20 (GM-CSF) antibody (in this case Ab-X). The background signal for the assay is defined as the signal obtained in wells with the coated antibody (in this case Ab-X), second solution phase antibody (in this case Ab-Y), buffer only (i.e. no CD20 (GM-CSF)) and CD20 (GM-CSF) detection reagents. The positive control signal for the assay is defined as the signal obtained in wells with the coated antibody (in this case Ab-X), second solution phase antibody buffer only (i.e. no second solution phase antibody), CD20 (GM-CSF) and CD20 (GM-CSF) detection reagents. The ELISA assay needs to be run in such a manner so as to have the positive control signal be at least 6 times the background signal.

To avoid any artifacts (e.g. significantly different affinities between Ab-X and Ab-Y for CD20 (GM-CSF)) resulting from the choice of which antibody to use as the coating antibody and which to use as the second (competitor) antibody, the cross-blocking assay needs to be run in two formats: 1) format 1 is where Ab-X is the antibody that is coated onto the ELISA plate and Ab-Y is the competitor antibody that is in solution and 2) format 2 is where Ab-Y is the antibody that is coated onto the ELISA plate and Ab-X is the competitor antibody that is in solution.

1-15. (canceled)

16. A method for the treatment of a patient having a B cell malignancy comprising administering to said patient a therapeutically effective amount of a combination comprising an antibody specific for CD20 and an antibody specific for GM-CSF.

17. The method of claim 16, wherein said B cell malignancy is selected from non-Hodgkin's lymphoma, Burkitt's lymphoma, small lymphocytic lymphoma, primary effusion lymphoma, diffuse large B-cell lymphoma, splenic marginal zone lymphoma, MALT (mucosa-associated lymphoid tissue) lymphoma, hairy cell leukemia, chronic lymphocytic leukemia, B-cell prolymphocytic leukemia, B cell lymphomas (e.g. various forms of Hodgkin's disease, B cell non-Hodgkin's lymphoma (NHL), leukemias (e.g. acute lymphoblastic leukemia (ALL), chronic lymphocytic leukemia (CLL; also termed B cell chronic lymphocytic leukemia BCLL), hairy cell leukemia and chronic myoblastic leukemia) and myelomas (e.g. multiple myeloma).

18. The method of claim 16, wherein said antibody specific for CD20 binds to a polypeptide comprising the following amino acid sequence:
(SEQ ID NO: 10)
MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP.

19. The method of claim 18, wherein said antibody specific for CD20 is an antibody specific for CD20 comprising an HCDR1 region of sequence SYNMH (SEQ ID NO: 13), an HCDR2 region of sequence AIYPGNGDTSYNQKFKG (SEQ ID NO: 14), an HCDR3 region of sequence STYYGGDWYFNV (SEQ ID NO: 15), an LCDR1 region of sequence RASSSVSYIH (SEQ ID NO: 16), an LCDR2 region of sequence ATSNLAS (SEQ ID NO: 17). and an LCDR3 region of sequence QQWTSNPPT (SEQ ID NO: 18).

20. The method of claim 18, wherein said antibody specific for CD20 is an antibody which cross-competes with an antibody specific for CD20 comprising an HCDR1 region of sequence SYNMH (SEQ ID NO: 13), an HCDR2 region of sequence AIYPGNGDTSYNQKFKG (SEQ ID NO: 14), an HCDR3 region of sequence STYYGGDWYFNV (SEQ Ill NO: 15), an LCDR1 region of sequence RASSSVSYIH (SEQ ID NO: 16), an LCDR2 region of sequence ATSNLAS (SEQ ID NO: 17), and an LCDR3 region of sequence QQWTSNPPT (SEQ ID NO: 18).

21. The method of claim 16, wherein said antibody specific for GM-CSF binds to a polypeptide comprising the following amino acid sequence:
(SEQ ID NO: 7)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE.

22. The method of claim 18, wherein said antibody specific for GM-CSF is an antibody specific for GM-CSF comprising an HCDR1 region of sequence GFTFSSYWMN (SEQ ID NO: 1), an HCDR2 region of sequence GIENKYAGGATYYAASVKG (SEQ ID NO: 2), an HCDR3 region of sequence GFGTDF (SEQ TD NO: 3), an LCDR1 region of sequence SGDSIGKKYAY (SEQ ID NO: 4), an LCDR2 region of sequence KKRPS (SEQ ID NO: 5), and an LCDR3 region of sequence SAWGDKGM (SEQ ID NO: 6).

23. The method of claim 18, wherein said antibody specific for GM-CSF is an antibody which cross-competes with an antibody specific for GM-CSF comprising an HCDR1 region of sequence GFTFSSYWMN (SEQ ID NO: 1), an HCDR2 region of sequence GIENKYAGGATYYAASVKG (SEQ ID NO: 2), an HCDR3 region of sequence GFGTDF (SEQ ID NO: 3), an LCDR1 region of sequence SGDSIGKKYAY (SEQ ID NO: 4), an LCDR2 region of sequence KKRPS (SEQ ID NO: 5), and an LCDR3 region of sequence SAWGDKGM (SEQ ID NO: 6).

24. A method for the treatment of a patient having an inflammatory disorder comprising administering to said patient therapeutically effective amount of a combination comprising an antibody specific for CD20 and an antibody specific for GM-CSF.

25. The method of claim 24, wherein said inflammatory disorder is selected from ulcerative colitis, Crohn's disease, inflammatory bowel disease, rheumatoid arthritis, myositis, multiple sclerosis, neuromyelitis optica, atherosclerosis, psoriasis, systemic lupus erythematosus, nephritis, glomerulonephritis, autoimmune hepatobiliary disease, graft-versus-host disease, atopic dermatitis, asthma, neurodegenerative disease (e.g., Alzheimer's disease), demyelinating polyradiculopathy, neuropathic pain, atherosclerosis, age-related macular degeneration, diabetic nephropathy, sarcoidosis-origined uveitis, or diabetes mellitus.

26. The method of claim 24, wherein said antibody specific for CD20 binds to a polypeptide comprising the following amino acid sequence:
(SEQ ID NO: 10)
MTTPRNSVNGTFPAEPMKGPIAMQSGPKPLFRRMSSLVGPTQSFFMRESK
TLGAVQIMNGLFHIALGGLLMIPAGIYAPICVTVWYPLWGGIMYIISGSL
LAATEKNSRKCLVKGKMIMNSLSLFAAISGMILSIMDILNIKISHFLKME
SLNFIRAHTPYINIYNCEPANPSEKNSPSTQYCYSIQSLFLGILSVMLIF
AFFQELVIAGIVENEWKRTCSRPKSNIVLLSAEEKKEQTIEIKEEVVGLT
ETSSQPKNEEDIEIIPIQEEEEEETETNFPEPPQDQESSPIENDSSP.

27. The method of claim 26, wherein said antibody specific for CD20 is an antibody specific for CD20 comprising an HCDR1 region of sequence SYNMH (SEQ ID NO: 13), an HCDR2 region of sequence AIYPGNGDTSYNQKFKG (SEQ ID NO: 14), an HCDR3 region of sequence STYYGGDWYFNV (SEQ ID NO: 15), an LCDR1 region of sequence RASSSVSYIH (SEQ ID NO: 16), an LCDR2 region of sequence ATSNLAS (SEQ ID NO: 17), and an LCDR3 region of sequence QQWTSNPPT (SEQ ID NO: 18).

28. The method of claim 26, wherein said antibody specific for CD20 is an antibody which cross-competes with an antibody specific for CD20 comprising an HCDR1 region of sequence SYNMH (SEQ ID NO: 13), an HCDR2 region of sequence AIYPGNGDTSYNQKFKG (SEQ ID NO: 14), an HCDR3 region of sequence STYYGGDWYFNV (SEQ ID NO: 15), an LCDR1 region of sequence RASSSVSYIH (SEQ ID NO: 16), an LCDR2 region of sequence ATSNLAS (SEQ ID NO: 17), and an LCDR3 region of sequence QQWTSNPPT (SEQ ID NO: 18).

29. The method of claim 24, wherein said antibody specific for GM-CSF binds to a polypeptide comprising the following amino acid sequence:
(SEQ ID NO: 7)
MWLQSLLLLGTVACSISAPARSPSPSTQPWEHVNAIQEARRLLNLSRDTA
AEMNETVEVISEMFDLQEPTCLQTRLELYKQGLRGSLTKLKGPLTMMASH
YKQHCPPTPETSCATQIITFESFKENLKDFLLVIPFDCWEPVQE.

30. The method of claim 26, wherein said antibody specific for GM-CSF is an antibody specific for GM-CSF comprising an HCDR1 region of sequence GFTFSSYWMN (SEQ ID NO: 1), an HCDR2 region of sequence GIENKYAGGATYYAASVKG (SEQ ID NO: 2), an HCDR3 region of sequence GFGTDF (SEQ ID NO: 3), an LCDR1 region of sequence SGDSIGKKYAY (SEQ ID NO: 4), an LCDR2 region of sequence KKRPS (SEQ ID NO: 5), and an LCDR3 region of sequence SAWGDKGM (SEQ ID NO: 6).

31. The method of claim 26, wherein said antibody specific for GM-CSF is an antibody which cross-competes with an antibody specific for GM-CSF comprising an HCDR1 region of sequence GFTFSSYWMN (SEQ ID NO: 1), an HCDR2 region of sequence GIENKYAGGATYYAASVKG (SEQ ID NO: 2), an HCDR3 region of sequence GFGTDF (SEQ ID NO: 3), an LCDR1 region of sequence SGDSIGKKYAY (SEQ ID NO: 4), an LCDR2 region of sequence KKRPS (SEQ ID NO: 5), and an LCDR3 region of sequence SAWGDKGM (SEQ ID NO: 6).
